(19) United States
(12) Patent Application Publication
(10) Pub. No.: US 2019/0021999 A1
(43) Pub. Date: Jan. 24, 2019

(54) EXTREMELY SMALL NANOPARTICLES OF DEGRADABLE POLYMERS

(71) Applicant: Phosphorex, Inc., Hopkinton, MA (US)

(72) Inventor: Bin Wu, Lexington, MA (US)

(21) Appl. No.: 15/897,371

(22) Filed: Feb. 15, 2018

Related U.S. Application Data
(63) Continuation of application No. PCT/US2016/047245, filed on Aug. 17, 2016.
(60) Provisional application No. 62/206,003, filed on Aug. 17, 2015.

(51) Int. Cl. A61K 9/113; A61K 47/34; A61K 9/16

(52) U.S. Cl. CPC A61K 9/113; A61K 9/1635; A61K 47/34

(57) ABSTRACT

The disclosure provides methods of manufacturing small nanoparticles of degradable polymers.

### REFERENCE TO RELATED APPLICATIONS

This application is a continuation of International Application No. PCT/US2016/047245, which designated the United States and was filed on Aug. 17, 2016, published in English, which claims the benefit of the filing date under 35 U.S.C. 119(e) to U.S. Provisional Application No. 62/206,003, filed on Aug. 17, 2015, the entire contents of which are incorporated herein by reference.

### BACKGROUND OF THE INVENTION

Particles, especially nanoparticles prepared from degradable and absorbable polymers, especially biodegradable polymers such as polylactic acid, polylactide, poly(lactide-co-glycolide) (PLGA), polycaprolactone, polyanhydrides, polyorthoesters, poly(hydroxyalkanoate)s, poly(sebacic acid), polyphosphoesters, and polyphosphazenes are very useful in the biomedical research, development and manufacturing. For example, microspheres and nanoparticles of PLGA polymers have already been produced worldwide and are used in many applications, including drug delivery. Various types of active pharmaceutical ingredients (APIs) can be encapsulated into biodegradable and bio-absorbable particles for controlled release and targeted drug delivery. These particles can be made as large as hundreds of microns or as small as hundreds of nanometers; however, it is still a challenge to prepare and load drugs into polymer nanoparticles under 200 nm, especially under 100 nm. One of the reasons small nanoparticles are difficult to make is because small particles have extremely high surface energies, which make them very easy to combine, or aggregate, into larger particles. It also takes tremendous mechanical forces to break polymer materials into such small sized particles. Suitable surfactants and surface stabilizers may also be needed in the process of manufacturing, during storage and in the application of said extremely small nanoparticles; therefore, there is a need to develop a formula and process for the preparation of small nanoparticles of biodegradable and bioabsorable polymers, and for the encapsulation of APIs into such small nanoparticles.

Recently, a precipitation method for creating PLGA nanoparticles as small as 5 nm was developed in which a microfluidic system is utilized (PCT/US2007/071901, 2010); however, this method can only be used to prepare plain PLGA nanoparticles without any API encapsulated therein, or PLGA nanoparticles encapsulating only solvent soluble drugs. It is not feasible to encapsulate water-soluble drugs into PLGA nanoparticles using this method. This poses a problem because many important drug molecules are water soluble and it is desirable to be able to encapsulate these drug molecules into small nanoparticles of biodegradable polymers. For example, most biologic APIs including proteins, peptides, antibodies, enzymes, growth factors, oligoneucliotides, DNA's, RNA's are water soluble. For these reasons, there is a need to develop a different method and process for the synthesis of nanoparticles of biodegradable and bioabsorable polymers that can create extremely small particles while allowing for the encapsulation of water soluble, hydrophilic drug molecules in addition to the encapsulation of hydrophobic, water insoluble drugs.

### SUMMARY OF THE INVENTION

The invention described herein is partly based on the realization that, to encapsulate a water-soluble drug, a double emulsion process is commonly used, which requires the polymer (e.g., PLGA polymer) to be dissolved in a water immiscible solvent, whereas the solvents used in the precipitation method are generally water miscible. Thus, to solve this problem, the current invention provides a method of synthesizing small polymeric nanoparticles by utilizing a single emulsion technique, described herein. This invention also provides a method of synthesizing small polymeric nanoparticles by utilizing a double emulsion technique, described herein. This invention also provides a method of synthesizing small polymeric nanoparticles loaded with therapeutic agents by utilizing a single emulsion technique, described herein. This invention also provides a method of synthesizing small polymeric nanoparticles loaded with therapeutic agents by utilizing a double emulsion technique, described herein. This invention also provides a composition of small polymeric nanoparticles, described herein. This invention also provides a composition of small polymeric nanoparticles loaded with therapeutic agents, described herein. This invention also provides a composition of small polymeric nanoparticles coated with non-therapeutic agents, described herein.

More specifically, the invention described herein provides a method for preparing polymeric nanoparticles, wherein said method comprises a single emulsion process comprising: (a) dissolving a pharmaceutically acceptable polymer in a first solvent to form a polymer solution; (b) emulsifying the polymer solution in a second solvent to form an emulsion, wherein the first solvent is not miscible or only partially miscible with the second solvent; and (c) removing the first solvent to form said nanoparticles, wherein said nanoparticles have an Z average particle size of about 150 nm or less.

In one embodiment, the particles are microparticles or nanoparticles. For example, the particles may be nanoparticles. Optionally, the nanoparticles have average particle sizes selected from the group consisting of from about 1 nm to about 150 nm, from about 10 nm to about 100 nm, and from about 20 nm to about 90 nm.

In one embodiment, the method described above also includes the step of dissolving or suspending an active pharmaceutical ingredient (API) in the first solvent before emulsification (step (b) above). In certain embodiments, the API is a hydrophilic, amphiphilic or hydrophobic drug. In certain embodiments, the API is a biologic entity. For example, the biologic entity can be selected from the group consisting of: proteins, peptides, growth factors, oligonucleotides, antibodies, polycarbohydrates, enzymes, amino acids, DNA, RNA, and ligands.

In certain embodiments, the method described above also includes the step of dissolving a pharmaceutically acceptable polymer with the therapeutic agent in the first solvent before emulsification.

In certain embodiments, the method further comprises dissolving or dispersing an API in the first solvent before emulsification.

In certain embodiments, the API is soluble in the first solvent.

In certain embodiments, the polymer solution further comprises a surfactant.

In certain embodiments, a surfactant is optionally dissolved in the second solvent before emulsification.

In certain embodiments, the second solvent optionally contains a fraction of the first solvent. For example, the second solvent may be saturated with the first solvent.

In another embodiment, the method described above also includes the step of adding a third solvent and emulsifying again in the presence of the third solvent after the first emulsification to form a second emulsion, but before removing the first solvent.

Thus in a related aspect, the invention also provides a method for preparing polymeric nanoparticles, wherein said method comprises a double emulsion process comprising: (a) dissolving a pharmaceutically acceptable polymer in a first solvent to form a polymer solution; (b) adding a small amount (e.g., 0.5% (v/v), 1% (v/v), 5% (v/v)) of a second solvent to the polymer solution to form a mixture, wherein the first solvent is not miscible or only partially miscible with the second solvent; (c) emulsifying the mixture to form a first emulsion; (d) emulsifying the first emulsion in a third solvent to form a second emulsion; and, (e) removing the first solvent to form said nanoparticles, wherein said nanoparticles have an Z average particle size of about 150 nm or less.

In certain embodiments, the second and third solvents are the same solvent, and optionally, said same solvent is water.

In certain embodiments, the third solvent optionally contains a fraction of the second solvent. For example, the third solvent may be saturated with the second solvent.

In certain embodiments, the third solvent further comprises a surfactant. Optionally, the surfactant is selected from the group consisting of detergents, wetting agents, emulsifiers, foaming agents, and dispersants. Optionally, the surfactant is polyvinyl alcohol.

In certain embodiments, the method further comprises dissolving or dispersing an active pharmaceutical ingredient (API) in the first solvent before emulsification. In certain embodiments, the API is soluble in the first solvent.

In certain embodiments, the method further comprises dissolving or dispersing an API in the second solvent before emulsification. In certain embodiments, the API is soluble in the second solvent. In certain embodiments, the API is a biologic entity. For example, the biologic entity may be selected from the group consisting of a protein, a peptide, a growth factor, an oligonucleotide, an antibody, a polycarbohydrate, an enzyme, an amino acid, a DNA, an RNA, and a ligand.

In certain embodiments, the method further comprises dissolving or dispersing a first API in the first solvent and dissolving or dispersing a second API in the second solvent. In certain embodiments, the first API is soluble in the first solvent. In certain embodiments, the second API is soluble in the second solvent. In certain embodiments, the second API is a biologic entity.

In certain embodiments, the emulsification is performed using a method selected from the group consisting of sonication, stirring, homogenization, microfluidization and combination thereof. In one embodiment, the emulsification is performed using microfluidization. In certain embodiments, the microfluidization is performed at an applied pressure selected from the group consisting of 1-100,000 psi, 1,000-70,000 psi, and 5,000-30,000 psi. In certain embodiments, the microfluidization is performed at a flow rate of 1 mL/min-100 L/min, preferably 1 mL/min-1 L/min. In certain embodiments, the emulsion is cycled through the microfluidizer 1-100 times, preferably 2-10 times.

In certain embodiments, the pharmaceutically acceptable polymer is selected from the group consisting of: PLA, PLGA, PEG-PLGA copolymer, PEG-PLA copolymer, PEG-PGA copolymer, poly(ethylene glycol), polycaprolactone, polyanhydrides, poly(ortho esters), polycyanoacrylates, poly(hydroxyalkanoate)s, poly(sebasic acid), polyphosphazenes, polyphosphoesters, modified poly(saccharide)s, mixtures and copolymers thereof.

In another embodiment, the pharmaceutically acceptable polymer is PLGA, and copolymers of PLGA such as PEG-PLGA.

In certain embodiments, the pharmaceutically acceptable polymer optionally comprises functional groups. For example, the functional groups may be selected from the group containing carboxyl, amino, diamine, thiol, aldehyde, hydroxysuccinimide ester, dihydrazide, hydroxysuccinimide-sulfonic acid, maleimide, and azide.

In certain embodiments, a dye or pigment can be incorporated into the nanoparticles to facilitate the imaging of the particles.

In certain embodiments, the method further comprises adsorbing or conjugating biologic or chemical entities to the surface of said nanoparticles.

In another embodiment, the first solvent is not miscible with water and is selected from the group containing ethyl acetate, dichloromethane, and chloroform. Optionally a water-miscible solvent can be mixed with the non-water miscible solvent as a co-solvent for the dissolution of the polymer or the API or both. In another embodiment, the second solvent is ethanol or water. In one aspect of this embodiment, the second solvent is water. In another embodiment, the third solvent is ethanol or water. In one aspect of this embodiment, the third solvent is water.

In certain embodiments, the first solvent optionally is mixed with a co-solvent. The co-solvent may be miscible with water.

In another embodiment, the polymer solution has a concentration selected from the group consisting of 1 μg/mL-1 g/mL percent by weight, 1 mg/mL-500 mg/mL percent by weight, and 10 mg/mL-100 mg/mL percent by weight.

Another aspect of the invention provides a polymeric nanoparticle produced according to any one of the methods of the invention.

A related aspect of the invention provides a polymeric nanoparticle, comprising: 0-100% of pharmaceutically acceptable polymer (such as PLGA), 0-100% PEGylated polymer (such as PEG-PLGA), 0-100% functionalized polymer (such as functionalized PLGA or functionalized PEG-PLGA), and 0-50% of fluorescent polymer derivative (such as fluorescent-PLGA derivative).

In certain embodiments, the polymeric nanoparticle comprises: 0-70% of pharmaceutically acceptable polymer (such as PLGA), 0-50% PEGylated polymer (such as PEG-PLGA), 0-30% functionalized polymer (such as functionalized PLGA or functionalized PEG-PLGA), and 0-30% of fluorescent polymer derivative (such as fluorescent-PLGA derivative).

In certain embodiments, the polymeric nanoparticles of the invention further comprises at least one API.

It should be understood that one of skill in the art can readily combine any one embodiment described herein, including the specific examples below, with any other embodiment(s) of the invention within the spirits of the invention.

### DETAILED DESCRIPTION OF THE INVENTION

Reference will now be made in detail to certain embodiments. While enumerated embodiments will be described, it will be understood that they are not intended to limit the invention to those embodiments. On the contrary, the invention is intended to cover all alternatives, modifications, and equivalents. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### Definitions

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “nanoparticles” is used herein to describe roughly round, sphere, or sphere-like in shape, and are generally within the size range of, e.g., between about 1-1,000 nm, between about 10-1,000 nm, between about 50-1,000 nm, between about 100-500 nm, and between 1-200 nm. The subject nanoparticles may also include particles that are less likely to clump in vivo.

The term “mammal” as used herein includes (but is not limited to) guinea pigs, dogs, cats, rats, mice, hamsters, non-human primates, and may also include human. In certain embodiments, the mammal has or is at risk of developing a disease described herein.

The phrase “pharmaceutically acceptable” indicates that the substance or composition is compatible chemically and/or toxicologically, with the other ingredients comprising a composition, and the mammal being treated therewith.

The term “biodegradable” refers to materials, more specifically polymers, having the capability to decompose within the body of a mammal without causing substantial toxic effects.

The phrase “extremely small” refers to polymeric particles with sizes on the nanometer scale, preferably smaller than 100 nm.

The phrase “PLGA” refers to poly(lactide-co-glycolide) or the derivatives, mixtures or copolymers thereof.

### Microfluidics

Microfluidics is the science and technology of manipulating flows in microscale channels, typically ranging from 10 μm-100 μm in size (Whitesides, 2006, Nature, 442:368, incorporated herein by reference). Using microfluidics, time consuming, laborious steps in chemical and biological analysis could be efficiently carried out in miniaturized channels and chambers with faster throughput and time-to-result, smaller sample consumption, and lower cost (Manz et al. 1992, J. Chromatography, 593:253). Microfluidics offers advantages that have made it a very useful tool for particle synthesis (deMello and deMello, 2004, Lab on a Chip, 4: 1 IN)-(i) the ability to rapidly mix reagents and provide homogeneous reaction conditions, (ii) continuous variation of reaction parameters, and (iii) addition of reactants at precise time intervals.

In general, a microfluidic device comprises at least two channels that converge into a mixing apparatus. In some embodiments, the channels join together at an angle ranging between zero degrees and 180 degrees. A stream of fluid is capable of flowing through each channel, and the streams join and flow into the mixing apparatus. In general, at least one stream comprises a polymeric solution, and at least one stream comprises a non-solvent. In some embodiments, the flow of the streams is laminar.

In some embodiments, the channels have a circular cross-section. In some embodiments, the channels that converge into the mixing apparatus are of uniform shape. In some embodiments, the width or height of each channel ranges from approximately 1 μm to approximately 1000 μm. In some embodiments, the length of each channel ranges from approximately 100 μm to approximately 10 cm. Channels may be composed of any material suitable for the flow of fluid through the channels. Typically, the material is one that is resistant to solvents and non-solvents that are used in the preparation of particles. In general, the material is not one that will dissolve or react with the solvent or non-solvent. In some embodiments, channels are composed of glass, silicon, metal, metal alloys, polymers, plastics, photocurable epoxy, ceramics, or combinations thereof. In some embodiments, channels are formed by lithography, etching, embossing, or molding of a polymeric surface. In general, the fabrication process may involve one or more of any of the processes described herein, and different parts of a device may be fabricated using different methods and assembled or bonded together.

Typically, a source of fluid is attached to each channel, and the application of pressure to the source causes the flow of the fluid in the channel. The pressure may be applied by a syringe, a pump, and/or gravity. In some embodiments, the applied pressure can be regulated (i.e. the applied pressure may be increased, decreased, or held constant). In some embodiments, the flow rate can be regulated by adjusting the applied pressure. In some embodiments, the flow rate can be regulated by adjusting the size (e.g. length, width, and/or height) of the channel. In some embodiments, the flow rate may range from 0.001 ml/min to 100 l/min, preferably from 1 ml/min to 10 ml/min.

In some embodiments, the same amount of pressure is applied to all of the channels and/or inlet streams. In some embodiments, different amounts of pressure are applied to different channels and/or inlet streams. Thus, in some embodiments, the flow rate may be the same through all channels and/or inlet streams, or the flow rate may be different in different channels and/or inlet streams.

In some embodiments, the emulsion is cycled through the microfluidizer multiple times. Preferably, the emulsion is cycled through the microfluidizer 1-100 times, or 2-10 times.

### Synthesis Methods and Compositions

Provided herein are synthesis methods for creating extremely small (e.g., 150 nm or less, or 100 nm or less) polymeric nanoparticles, and the compositions of said nanoparticles. More specifically the current invention provides a method of synthesizing small polymeric nanoparticles by utilizing a single emulsion technique, described herein. This invention also provides a method of synthesizing small polymeric nanoparticles by utilizing a double emulsion technique, described herein. This invention also provides a method of synthesizing small polymeric nanoparticles loaded with therapeutic agents by utilizing a single emulsion technique, described herein. This invention also provides a method of synthesizing small polymeric nanoparticles loaded with therapeutic agents by utilizing a double emulsion technique, described herein. This invention also provides a composition of small polymeric nanoparticles, described herein. This invention also provides a composition of small polymeric nanoparticles loaded with therapeutic agents, described herein. This invention also provides a composition of small polymeric nanoparticles coated with non-therapeutic agents, described herein.

In certain embodiments, the nanoparticles are comprised of a pharmaceutically acceptable biodegradable and bioabsorable polymer.

Pharmaceutically acceptable biodegradable and bioabsorable polymers used in certain embodiments include, but are not limited to water soluble polymers including poly (acrylic acid), poly (ethylene oxide), poly (ethylene glycol), poly (vinyl pyrrolidone), poly (vinyl alcohol), polyacrylamide, poly (isopropyl acrylamide), and poly (cyclopropyl methacryl amide); cellulose-based polymers including ethyl cellulose, carboxymethyl cellulose, hydroxyethyl cellulose, hydroxypropyl cellulose, hydroxypropyl methyl cellulose, and cellulose acetate phthalate; hydrocolloids including alginic acid, carrageenan, chitosan, hyaluronic acid, and pectinic acid; water-insoluble polymers including (lactide-co-glycolide) polymers; starch-based polymers including starch and sodium starch glycolate; polycaprolactone, polyanhydrides, poly(ortho esters), polycyanoacrylates, poly(hydroxyalkanoate)s, polyphosphazenes, polyphosphoesters, modified poly(saccharide)s, plastics including polycarbonate, poly (vinyl acetate), polypropylene, polyethylene, poly(hydroxyethyl methacrylate), acrylic acid and butyl acrylate copolymer, 2-ethylhexyl acrylate and butyl acrylate copolymer, and polyethylene and polyethylene terephthalate, the mixtures and copolymers thereof.

In certain embodiments, said pharmaceutically acceptable biodegradable polymer is PLGA with a lactide to glycolide (L/G) ratio ranging from 100/0 to 0/100, 95/5 to 5/95, 85/15 to 15/85, 75/25 to 25/75, 65/35 to 35/65, or 50/50.

In certain embodiments, said PLGA polymer is a PLGA-PEG copolymer.

In certain embodiments, said PLGA-PEG copolymer is a diblock or a triblock copolymer of PLGA and PEG.

In certain embodiments, said PLGA polymer or copolymer is further functionalized with groups selected from the group comprising carboxyl, amino, diamine, thiol, aldehyde, hydroxysuccinimide ester, dihydrazide, hydroxysuccinimide-sulfonic acid, maleimide, and azide. Said functional groups may be incorporated by introducing to the nanoparticle formulation a polymer derivative containing the functional groups on the polymer molecule. Examples of such polymer derivatives include AI016 (PLGA-NH₂), AI025 (PLGA-SH), AI096 (PLGA-NHS), AI052 (PLGA-PEG-Mal), AI078 (PLGA-PEG-COOH), AI087 (PCL-NH₂), AI021 (NH₂-PLGA-NH₂), AI086 (mPEG-PLGA-NH₂), all of which may be found on the website of Polyscitech of West Lafayette, IN (https://akinainc.com/polyscitech/products/polyvivo/catalogue.php#PolymerVisualization).

The functional groups can be utilized to facilitate the chemical conjugation of certain ligands onto the small nanoparticles of the current invention. Said ligands can be a small molecule or biologic therapeutic agent, or non-therapeutic agent.

In certain embodiments, a dye or pigment can be incorporated into the nanoparticles to facilitate the imaging of the particles. Said dye or pigment may be visibly colored, fluorescent, phosphorescent, or luminescent. The dye or pigment may be encapsulated into the polymeric nanoparticles by dissolving it in the initial polymer solution in a single or double emulsion process. Alternatively, the dye or pigment can be dissolved in the aqueous solution with the hydrophilic API in a double emulsion process.

Said dye or pigment may also be incorporated by adding a polymer derivative that contains the dye or pigment chemically bound on the polymer. Such polymer-dye conjugates are commercially available. For example, AV017 (mPEG-PLA-FKR648), AV18 (mPEG-PLGA-FKR560), AV016 (PLA-Fluorescein), AV013 (PLA-FPR648), AV015 (PLGA-FKR648), AV001 (PLGA-Fluorescein), AV006 (PLGA-FPI749), and AV011 (PLGA-Rhodamine B) can all be found on the website of Polyscitech of West Lafayette, IN (https://akinainc.com/polyscitech/products/polyvivo/catalogue.php#PolymerVisualization).

In certain embodiments, said pharmaceutically acceptable biodegradable polymer can be a derivative, a copolymer or a mixture of the above-mentioned polymers.

In certain embodiments, the emulsifying steps to form the single or double emulsions comprise homogenization, mechanical stirring, microfluidization or combination thereof.

In certain embodiments, the emulsion process is a single emulsion process and comprises: (1) dissolving the pharmaceutically acceptable polymer in a first solvent to form a polymer solution; (2) emulsifying the polymer solution in a second solvent to form an emulsion, wherein the first solvent is not miscible or only partially miscible with the second solvent; and, (3) removing the first solvent to form said nanoparticles.

As used herein, partially miscible may refer to the fact that no more than 80% (v/v), 70% (v/v), 60% (v/v), 50% (v/v), 40% (v/v), 30% (v/v), 20% (v/v), 10% (v/v), 5% (v/v), 2% (v/v), or 1% (v/v) of the first solvent is miscible with the second solvent.

In certain embodiments, the resulting nanoparticles have an average particle size of about 150 nm or less.

In certain embodiments, the average particle size is measured by any one of the following: Z average particle size, volume average particle size, and number average particle size. In certain embodiments, the average particle size is measured by Z average particle size.

In certain embodiments, the measurements are based on substantially the same condition as described in one of the examples herein.

In certain embodiments, a fluorescent dye can be optionally added to the polymer solution before the emulsification step.

In certain embodiments, a therapeutic agent is co-dissolved in the first solvent together with said pharmaceutically acceptable polymer in the above nanoparticle fabrication process.

In certain embodiments, the emulsion process is a double emulsion process comprising: (1) dissolving the pharmaceutically acceptable polymer and optionally an active pharmaceutical ingredient (API) in a first solvent to form a polymer solution; (2) adding a small amount (e.g., about 0.01-50% (v/v), about 0.05-25% (v/v), about 0.1-10% (v/v)) of a solution of a second solvent to the polymer solution to form a mixture, wherein the first solvent is not miscible or only partially miscible with the second solvent, and wherein the solution of the second solvent optionally comprises an API; (3) emulsifying the mixture to form a first emulsion; (4) emulsifying the first emulsion in the solution of a third solvent to form a second emulsion, wherein the solution of the third solvent optionally comprises a surfactant or emulsifier; and, (5) removing the first solvent to form said nanoparticles.

In certain embodiments, the resulting nanoparticles have an average particle size of about 150 nm or less.

In certain embodiments, the average particle size is measured by any one of the following: Z average particle size, volume average particle size, and number average particle size. In certain embodiments, the average particle size is measured by Z average particle size.

In certain embodiments, the measurements are based on substantially the same condition as described in one of the examples herein.

In certain embodiments, a fluorophor can be optionally added to the polymer solution in the above-mentioned double emulsion process.

The emulsification steps can be carried out by various methods including sonication, stirring, homogenization, microfluidization or combination thereof. However, the microfluidization is the preferred method in the current invention.

In certain embodiments, the applied pressure of the microfluidization device is from 1-100,000 psi, preferably from 1,000-70,000 psi, and more preferably 5,000-50,000 psi.

In the emulsifying step, the (aqueous) solution may contain a surfactant or surface stabilizer. Surfactants generally include compounds that lower the surface tension of a liquid, the interfacial tension between two liquids, or that between a liquid and a solid. Surfactants may act as detergents, wetting agents, emulsifiers, foaming agents, and dispersants. Surfactants are usually organic compounds that are amphiphilic, which contain both hydrophobic groups (usually branched, linear, or aromatic hydrocarbon chain(s), fluorocarbon chain(s), or siloxane chain(s) as “tail(s)”) and hydrophilic groups (usually heads).

While not wishing to be bound by any particular theory, surfactant may be useful for the formation and stabilization of the emulsion droplets. The surfactant may also comprise organic or inorganic pharmaceutical excipients, various polymers, oligomers, natural products, nonionic, cationic, zwitterionic, or ionic surfactants, and mixtures thereof.

The surfactants that can be used for the preparation of the subject (PLGA) microparticles/nanoparticles include polyvinyl alcohol, polyvinylpyrrolidone, Tween series, Pluronic series, Poloxamer series, Triton X-100, etc. Additional suitable surfactants are provided herein below.

Combinations of more than one surfactant can be used in the invention. Useful surfactants or surface stabilizers which can be employed in the invention may include, but are not limited to, known organic and inorganic pharmaceutical excipients. Such excipients include various polymers, low molecular weight oligomers, natural products, and surfactants. Surfactants or surface stabilizers include nonionic, cationic, zwitterionic, and ionic surfactants.

Representative examples of other useful surfactants or surface stabilizers include hydroxypropyl methylcellulose, hydroxypropylcellulose, polyvinylpyrrolidone, sodium lauryl sulfate, sodium dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available TWEENS® such as e.g., TWEEN 20® and TWEEN 80® (ICI Specialty Chemicals)); polyethylene glycols (e.g., CARBOWAXS 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., PLURONICS F68® and F108®, which are block copolymers of ethylene oxide and propylene oxide); poloxamines (e.g., TETRONIC 908®, also known as POLOXAMINE 908®, which is a tetrafunctional block copolymer derived from sequential addition of propylene oxide and ethylene oxide to ethylenediamine (BASF Wyandotte Corporation, Parsippany, N.J.)); TETRONIC 1508® (T-1508) (BASF Wyandotte Corporation), Tocopheryl polyethylene glycol succinate (TPGS), TRITONS X-200®, which is an alkyl aryl polyether sulfonate (Rohm and Haas); CRODESTAS F-110®, which is a mixture of sucrose stearate and sucrose distearate (Croda Inc.); p-isononylphenoxypoly-(glycidol), also known as OLIN-LOG® or SURFACTANT 10-G® (Olin Chemicals, Stamford, Conn.); Crodestas SL-40(Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-p-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

Examples of useful cationic surfactants or surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt) (also known as DPPE-PEG(2000)-Amine Na) (Avanti Polar Lipids, Alabaster, A1), Poly(2-methacryloxyethyl trimethylammonium bromide) (Polysciences, Inc., Warrington, Pa.) (also known as S1001), poloxamines such as TETRONIC 908®, also known as POLOXAMINE 908®, which is a tetrafunctional block copolymer derived from sequential addition of propylene oxide and ethylene oxide to ethylenediamine (BASF Wyandotte Corporation, Parsippany, N.J.), lysozyme, long-chain polymers such as alginic acid, carrageenan (FMC Corp.), and POLYOX (Dow, Midland, Mich.).

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy) 4 ammonium chloride or bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl (C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surfactants or surface stabilizers and other useful cationic surfactants or surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990), each of which is incorporated by reference herein in its entirety.

Nonpolymeric cationic surfactants or surface stabilizers are any nonpolymeric compound, such as benzalkonium chloride, a carbonium compound, a phosphonium compound, an oxonium compound, a halonium compound, a cationic organometallic compound, a quaternary phosphorous compound, a pyridinium compound, an anilinium compound, an ammonium compound, a hydroxylammonium compound, a primary ammonium compound, a secondary ammonium compound, a tertiary ammonium compound, and quaternary ammonium compounds of the formula NR₁R₂R₃R₄(+). For compounds of the formula NR₁R₂R₃R₄(+): (i) none of R₁-R₄ are CH₃; (ii) one of R₁-R₄ is CH₃; (iii) three of R₁-R₄ are CH₃; (iv) all of R₁-R₄ are CH₃; (v) two of R₁-R₄ are CH₃, one of R₁-R₄ is C₆H₅CH₂, and one of R₁-R₄ is an alkyl chain of seven carbon atoms or less; (vi) two of R₁-R₄ are CH₃, one of R₁-R₄ is C₆H₅CH₂, and one of R₁-R₄ is an alkyl chain of nineteen carbon atoms or more; (vii) two of R₁-R₄ are CH₃ and one of R₁-R₄ is the group C₆H₅ (CH₂)ₙ, where n>1; (viii) two of R₁-R₄ are CH₃, one of R₁-R₄ is C₆H₅CH₂, and one of R₁-R₄ comprises at least one heteroatom; (ix) two of R₁-R₄ are CH₃, one of R₁-R₄ is C₆H₅CH₂, and one of R₁-R₄ comprises at least one halogen; (x) two of R₁-R₄ are CH₃, one of R₁-R₄ is C₆H₅CH₂, and one of R₁-R₄ comprises at least one cyclic fragment; (xi) two of R₁-R₄ are CH₃ and one of R₁-R₄ is a phenyl ring; or (xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surfactants or surface stabilizers are known pharmaceutical excipients and are described in detail in the Handbook of Pharmaceutical Excipients, published jointly by the

American Pharmaceutical Association and The Pharmaceutical Society of Great Britain (The Pharmaceutical Press, 2000), specifically incorporated by reference herein.

The surfactants or surface stabilizers are commercially available and/or can be prepared by techniques known in the art.

Combinations of more than one solvent can be used in the invention. Useful solvents which can be employed in the invention may include, but are not limited to, 1,4-dioxane, tetrahydrofuran (THF), diethylether, methylethylether, dimethylether, acetone, acetonitrile, dimethyl sulfoxide (DMSO), dimethylformamide (DMF), glyme, ethyl acetate, dichloromethane, chloroform, hexane, polyethylene glycol, glycerin, acids, and alcohols (e.g. methanol, ethanol, isopropanol, butanol, etc.). Use of a combination of solvents may aid in decreasing the size of the nanoparticles.

In certain embodiments, the first solvent is a volatile solvent.

In certain embodiments, the polymer is a PLGA polymer, PEG-PLGA copolymer, or combination thereof. The volatile solvent is methylene chloride, ethyl acetate, or chloroform, or combination thereof.

In certain embodiments, the volatile solvent can be optionally mixed with a co-solvent.

In certain embodiments, said co-solvent is a water-miscible solvent. Examples of such water-miscible co-solvents are acetone, methanol, ethanol, THF, DMSO, DMF, DMA, acetonitrile, ethylene glycol, etc.

In certain embodiments, the solution of the second solvent comprises a surfactant.

In certain embodiments, said solution of the second solvent is mixed with a fraction of the first solvent. The volume ratio of the first solvent to the second solvent in the mixture is determined by the solubility of the first solvent in the second solvent, and may be from about 0.1:99.9 to 50:50, preferably from 1:99 to 20:80. Preferably, said solution of the second solvent is saturated with the first solvent.

In certain embodiments, the second solvent is an alcohol or water.

In certain embodiments, the second solvent is water.

In certain embodiments, the removal of solvent is by evaporation using an evaporating device such as a rotavap. The evaporation process can be optionally carried out by applying vacuum and heat. The solvent removal can also be by simply stirring the emulsion in a ventilated hood for several hours.

In certain embodiments, the final emulsion produced in a single emulsion or a double emulsion process is mixed with a large amount of aqueous solution to “quench” the nanoparticle suspension followed by the evaporation of the organic solvent used to dissolve the pharmaceutically acceptable polymer. Optionally the quenching solution can be saturated with the solvent used to dissolve the polymer.

After the solvent is removed, the nanoparticle suspension needs to be washed to remove any surfactant and any unincorporated therapeutic agent in the suspension. A typical washing process includes centrifuge, ultrafiltration, cross flow filtration and dialysis.

Purified nanoparticles may be collected by a drying process such as lyophilization and spray drying.

In certain embodiments, purified nanoparticles are lyophilized.

In certain embodiments, the purified nanoparticle suspension contains certain cryoprotectant. Examples of such cryoprotectant include but not limit to manitol, glucose, trehalose, and sucrose. The concentration of the cryoprotectant in the nanoparticle suspension may be from 0.01% to 95%, preferably from 0.1% to 10%.

Alternatively, nanoparticles may be stored without drying. For example, purified nanoparticle suspension can be stored under frozen conditions. Appropriate cryoprotectantis added to the nanoparticle suspension before subjecting it to the freezing condition. The temperature of the environment where the nanoparticle suspension is stored is from 0° C. to −100° C., preferably from −20° C. to −85° C.

In certain embodiments, the nanoparticles have average particle sizes of from about 1 nm to about 1000 nm, preferably from about 10 nm to about 100 nm, more preferably from about 20 nm to about 90 nm.

As used herein, particle size can be determined by any conventional particle size measuring techniques well known to those skilled in the art. Such techniques include, for example, sedimentation field flow fractionation, photon correlation spectroscopy, light scattering, dynamic light scattering, light diffraction, and disk centrifugation.

In certain embodiments, the API is a hydrophilic drug. Examples of hydrophilic drugs include, but are not limited to, isoniazid, rifampicin, pyrazinamide, ethambutol, streptomycin, acyclovir, acetyl cysteine, acetylcholine chloride, alatrofloxacin, alendronate, amantadine hydrochloride, ambenomium, amifostine, amiloride hydrochloride, aminocaproic acid, amphiphilicin B, atenolol, atracurium besylate, atropine, azithromycin, aztreonam, bacitracin, becalermin, belladona, bepridil hydrochloride, bleomycin sulfate, calcitonin, calcitonin salmon, carboplatin, capecitabine, capreomycin sulfate, cefamandole nafate, cefazolin sodium, cefepime hydrochloride, cefixime, cefonicid sodium, cefoperazone, cefotetan disodium, cefotoxime, cefoxitin sodium, ceftizoxime, ceftriaxone, cefuroxime axetil, cephalexin, cephapirin sodium, chrionic gonadotropin, cidofovir, cisplatin, cladribine, clidinium bromide, clindamycin, ciprofloxacin, clondronate, colistimethate sodium, deforoxamine, denileukin diftitox, desmopressin, diatrizoate megluamine, dicyclomine, didanosine, dirithromycin, dopamine hydrochloride, dornase alpha doxacurium chloride, doxorubicin, editronate disodium, elanaprilat, enkephalin, enoxacin, enoxaprin sodium, ephedrine, epinephrine, erythromycin, esmol hydrochloride, famiciclovir, fludarabine, fluoxetine, ganciclovir, gentamycin, glucagon, glycopyrolate, heparin sodium, indinavir sulfate, insulin, lamivudine, leucovorin calcium, leuprolide acetate, levofloxacin, lincomycin, lobucavir, lomefloxacin, loracarbef, mannitol, methotrexate, methscopolamine, metformin hydrochloride, metroprolol, mezocillin sodium, mivacurium chloride, nedocromil sodium, neostigmine bromide, neostigmine methyl sulfate, neutontin, norfloxacin, octreotide acetate, ofloxacin, olpadronate, oxytocin, pamidronate disodium, pancuronium bromide, paroxetine, pefloxacin, pentamindine isethionate, pentostatin, pentoxifylline, periciclovir, pentagastrin, phentolamine mesylate, phenylalanine, physostigmine salicylate, piperacillin sodium, polymixin B sulfate, pralidoxine chloride, pramlintide, pregabalin, propofenone, propenthaline bromide, pyridostigmine bromide, residronate, ribavarin, rimantadine hydrochloride, salmetrol xinafoate, sincalide, solatol, somatostatin, sparfloxacin, spectinomycin, stavudine, streptozocin, suxamethonium chloride, tacrine hydrochloride, terbutaline sulfate, thiopbeta ticarcillin, tiludronate, timolol, trandolapril, trimetrexate gluconate, trospectinomycin, trovafloxacin, tubocurarine chloride, valaciclovir, valsartan, vasopressin, vecoronium bromide, vinblastin, vincristine, vinorelbine, warfarin sodium, zalcitabine, zanamavir, zolandronate and zidovudine.

In certain embodiments, the API is an amphiphilic drug. Examples of amphiphilic drugs include, but are not limited to amphiphilicin B, bupivacaine, ropivacaine, prilocaine, mepivacaine, tetrocaine, etidocaine, morphine, fentanyl, alfentanil and sulfentanil.

In certain embodiments, the API is a hydrophobic drug. Examples of hydrophobic drugs include, but are not limited to, abietic acid, aceglatone, acenaphthene, acenocournarol, acetohexamide, acetomeroctol, acetoxolone, acetyldigitoxins, acetylene dibromide, acetylene dichloride, acetylsalicylic acid, alantolactone, aldrin, alexitol sodium, allethrin, allylestrenol, allyl sulfide, alprazolam, aluminum bis(acetylsalicylate), ambucetamide, aminochlothenoxazin, aminoglutethimide, amyl. chloride, androstenediol, anethole trithone, anilazine, anthralin, Antimycin A, aplasmomycin, arsenoacetic acid, asiaticoside, asternizole, aurodox, aurothioglycanide, 8-azaguanine, azobenzene, baicalein, Balsam Peru, Balsam Tolu, barban, baxtrobin, bendazac, bendazol, bendroflumethiazide, benomyl, benzathine, benzestrol, benzodepa, benzoxiquinone, benzphetamine, benzthiazide, benzyl benzoate, benzyl cinnamate, bibrocathol, bifenox, binapacryl, bioresmethrin, bisabolol, bisacodyl, bis(chlorophenoxy)methane, bismuth iodosubgallate, bismuth subgallate, bismuth tannate, Bisphenol A, bithionol, bornyl, bromoisovalerate, bornyl chloride, bornyl isovalerate, bornyl salicylate, brodifacoum, bromethalin, broxyquinoline, bufexamac, butamirate, butethal, buthiobate, butylated hydroxyanisole, butylated hydroxytoluene, calcium iodostearate, calcium saccharate, calcium stearate, capobenic acid, captan, carbamazepine, carbocloral, carbophenothin, carboquone, carotene, carvacrol, cephaeline, cephalin, chaulmoogric acid, chenodiol, chitin, chlordane, chlorfenac, chlorfenethol, chlorothalonil, chlorotrianisene, chlorprothixene, chlorquinaldol, chromonar, cilostazol, cinchonidine, citral, clinofibrate, clofaziminc, clofibrate, cloflucarban, clonitrate, clopidol, clorindione, cloxazolam, coroxon, corticosterone, cournachlor, coumaphos, coumithoate cresyl acetate, crimidine, crufomate, cuprobam, cyamemazine, cyclandelate, cyclarbamate cymarin, cyclosporin A, cypermethril, dapsone, defosfamide, deltamethrin, deoxycorticocosterone acetate, desoximetasone, dextromoramide, diacetazoto, dialifor, diathymosulfone, decapthon, dichlofluani, dichlorophen, dichlorphenamide, dicofol, dicryl, dicumarol, dienestrol, diethylstilbestrol, difenamizole, dihydrocodeinone enol acetate, dihydroergotamine, dihydromorphine, dihydrotachysterol, dimestrol, dimethisterone, dioxathion, diphenane, N-(1,2-diphenylethyl)nicotinamide, 3,4-di-[1-methyl 6-nitro-3-indolyl]-1H-pyrrole-2,5-dione (MNIPD), dipyrocetyl, disulfamide, dithianone, doxenitoin, drazoxolon, durapatite, edifenphos, emodin, enfenamic acid, erbon, ergocorninine, erythrityl tetranitrate, erythromycin stearate, estriol, ethaverine, ethisterone, ethyl biscournacetate, ethylhydrocupreine, ethyl menthane carboxamide, eugenol, euprocin, exalamide, febarbamate, fenalamide, fenbendazole, fenipentol, fenitrothion, fenofibrate, fenquizone, fenthion, feprazone, flilpin, filixic acid, floctafenine, fluanisone, flumequine, fluocortin butyl, fluoxymesterone, fluorothyl, flutazolam, fumagillin, 5-furftiryl-5-isopropylbarbituric acid, fusaftmgine; glafenine, glucagon, glutethimide, glybuthiazole, griseofulvin, guaiacol carbonate, guaiacol phosphate; halcinonide, hematoporphyrin, hexachlorophene, hexestrol, hexetidine, hexobarbital, hydrochlorothiazide, hydrocodone, ibuproxam, idebenone, indomethacin, inositol niacinate, iobenzamic acid, iocetamic acid, iodipamide, iomeglamic acid, ipodate, isometheptene, isonoxin, 2-isovalerylindane-1,3-dione, josamycin, 11-ketoprogesterone, laurocapram, 3-O-lauroylpyridoxol diacetate, lidocaine, lindane, linolenic acid, liothyronine, lucensomycin, mancozeb, mandelic acid, isoamyl ester, mazindol, mebendazole, mebhydroline, mebiquine, melarsoprol, melphalan, menadione, menthyl valerate, mephenoxalone, mephentermine, mephenyloin, meprylcaine, mestanolone, mestranol, mesulfen, metergoline, methallatal, methandriol, methaqualone, methylcholanthrene, methylphenidate, 17-methyltestosterone, metipranolol, minaprine, myoral, naftalofos, naftopidil, naphthalene, 2-naphthyl lactate, 2-(2-naphthyloxy)ethanol, naphthyl salicylate, naproxen, nealbarbital, nemadectin, niclosamide, nicoclonate, nicomorphine, nifuroquine, nifuroxazide, nitracrine, nitromersol, nogalamycin, nordazepam, norethandrolone, norgestrienone, octaverine, oleandrin, oleic acid, oxazepam, oxazolam, oxeladin, oxwthazaine, oxycodone, oxymesterone, oxyphenistan acetate, paclitaxel, paraherquamide, parathion, pemoline, pentaerythritol tetranitrate, pentylphenol, perphenazine, phencarbamide, pheniramine, 2-phenyl-6-chlorophenol, phenthnethylbarbituric acid, phenyloin, phosalone, O-phthalylsulfathiazole, phylloquinone, picadex, pifamine, piketopfen, piprozolin, pirozadil, pivaloyloxymethyl butyrate, plafibride, plaunotol, polaprezinc, polythiazide, probenecid, progesterone, promegestone, propanidid, propargite, propham, proquazone, protionamide, pyrimethamine, pyrimithate, pyrvinium pamoate, quercetin, quinbolone, quizalofo-ethyl, rafoxanide, rapamycin, rescinnamine, rociverine, ronnel, salen, scarlet red, siccanin, simazine, simetride, simvastatin, sirolimus, sobuzoxane, solan, spironolactone, squalene, stanolone, sucralfate, sulfabenz, sulfaguanole, sulfasalazine, sulfoxide, sulpiride, suxibuzone, talbutal, terguide, testosterone, tetrabromocresol, tetrandrine, thiacetazone, thiocolchicine, thioctic acid, thioquinox, thioridazine, thiram, thymyl N-isoamylcarbamate, tioxidazole, tioxolone, tocopherol, tolciclate, tolnaftate, triclosan, triflusal, triparanol, ursolic acid, valinomycin, verapamil, vinblastine, vitamin A, vitamin D, vitamin E, xenbucin, xylazine, zaltoprofen, and zearalenone.

In certain embodiments, the API is a biologic entity. Examples of biologic entities include, but are not limited to proteins, peptides, growth factors, oligonucleotides, antibodies, polycarbohydrates, enzymes, amino acids, DNA, RNA, and ligands.

In certain embodiments, the concentration of the polymer solution is 1 μg/mL-1 g/mL percent by weight, preferably 1 mg/mL-500 mg/mL percent by weight, and most preferably 10 mg/mL-100 mg/mL percent by weight.

In certain embodiments, the nanoparticles can be coated with therapeutic or non-therapeutic agents for the purpose of property enhancement. Non-therapeutic agents can include, but are not limited to targeting ligands, imaging agents, and surfactants. Nanoparticles can be coated through the process of physical adsorption or through the process of chemical conjugation. Physical adsorption can be accomplished by techniques well known to those skilled in the art. Chemical conjugation is achieved by covalent bonding between at least one functional group on the polymeric component of the conjugate and at least one functional group on the therapeutic or non-therapeutic agent, typically to form an ester, amide, urethane, hydrazone, thioether, carbonate, azo, mine (Schiff s base), carbon-carbon or disulfide bond. One of the embodiments of the current invention provides certain functional groups on the nanoparticles. These functional groups can be utilized for the chemical conjugation. The linkage between the polymer and agent may be designed according to known principles to be biologically labile if necessary, such that the agent is chemically free to exhibit the desired effect. Suitable methods and reaction conditions for chemical coupling of a pharmaceutical and a polymer are summarized in reviews by R. Duncan et al., Encyclopedia of Controlled Drug Delivery, 2:786 (E. Mathiowitz, editor); and by Kopecek et al., Advances in Polymer Science, 1995 (112), 55-123.

### EXAMPLES

### Example 1

### Preparation of PLGA Nanoparticles by a Single Emulsion Process

Approximately 75 grams of 1 wt % Pluronic F-68 aqueous solution was mixed with approximately 6 grams of ethyl acetate. Such mixture was mixed with approximately 25 grams of 1 wt % PLGA (50:50) solution in ethyl acetate in a glass container. The mixture was homogenized by a rotor stator mixer at approximately 8,000 RPM for 1 minute. Such formed emulsion was further processed on a Microfluidizer processor through an F12Y (75 μm) interaction chamber nozzle at approximately 15,000 psi for three passes. An ice water bath was placed around the cooling coil to cool the sample after processing. After the processing, the sample was stirred magnetically in a hood to allow the evaporation of ethyl acetate.

After the solvent evaporation, the particle size of the nanoparticle obtained was measured on Malvern Zetasizer and the results were as the following:

Z Average Particle Size=51.48±11.54 nm

### Example 2

### Preparation of PLGA Nanoparticles by a Double Emulsion Process

About 50 mg of PLGA (50:50; average molecule weight 63,000) was dissolved in 50 ml ethyl acetate. 1 ml of de-ionized H20 (inner water phase) was added to the PLGA solution and homogenized on a rotor stator homogenizer at 10,000 RPM for 1 minute. It was then processed with a Microfluidizer processor at 6,000 psi for 1 pass to give a first emulsion. Separately, 300 ml of a 0.5% aqueous solution of polyvinyl alcohol was prepared. The first emulsion was added to the aqueous solution of polyvinyl alcohol and processed with a Microfluidizer processer at 6000 psi for 3 passes to form the second emulsion.

The second emulsion was stirred magnetically to evaporate the ethyl acetate. The nanoparticles obtained were found to have the following particle sizes:

Z Average Particle Size=111.3±45.66 nm

The nanoparticle suspension was washed by a cross-flow filtration device with a 0.05 μm pore size to remove the polyvinyl alcohol, concentrated and lyophilized.

### Example 3

### Preparation of BSA Loaded PLGA Nanoparticles by a Double Emulsion Process

About 50 mg of PLGA (50:50; average molecular weight 63,000) was dissolved in 50 ml ethyl acetate. 1 ml of 10 mg/ml BSA in de-ionized Hao (inner water phase) was added to the PLGA solution and homogenized on a rotor stator homogenizer at 10,000 RPM for 1 minute. It was then processed with a Microfluidizer processor at 6,000 psi for 1 pass to give a first emulsion. Separately, 300 ml of a 0.5% aqueous solution of polyvinyl alcohol was prepared. The first emulsion was added to the aqueous solution of polyvinyl alcohol and processed with a Microfluidizer processer at 6000 psi for 3 passes to form the second emulsion.

The second emulsion was stirred magnetically to evaporate the ethyl acetate. The nanoparticles obtained were found to have the following particle sizes:

Z Average Particle Size=144.4±73.64 nm

The nanoparticle suspension was washed by a cross-flow filtration device with a 0.05 μm pore size to remove the polyvinyl alcohol and unincorporated BSA.

### Example 4

### Preparation of PEG-PLGA Nanoparticles Loaded with BSA and Paclitaxel by a Double Emulsion Process

About 75 mg of carboxyl terminal PLGA (50:50; average molecular weight about 33,000), along with about 75 mg of mPEG-PLGA (Mw ˜5,000:10,000) and about 30 mg of paclitaxel was dissolved in 50 ml methylene chloride. 1 ml of 10 mg/ml BSA in de-ionized H2O (inner water phase) was added to the polymer/paclitaxel solution and homogenized on a rotor stator homogenizer at 10,000 RPM for 1 minute. It was then processed with a Microfluidizer processor at 10,000 psi for 5 pass to give a first emulsion. Separately, 300 ml of a 0.5% aqueous solution of polyvinyl alcohol was prepared. The first emulsion was added to the aqueous solution of polyvinyl alcohol and processed with a Microfluidizer processer at 8,000 psi for 3 passes to form the second emulsion.

The second emulsion was stirred magnetically to evaporate the solvent. The nanoparticles obtained were found to have the following particle sizes:

Z Average Particle Size=89.3±33.5 nm

The nanoparticle suspension was washed by a cross-flow filtration device with a 0.05 μm pore size to remove the polyvinyl alcohol and unincorporated BSA and paclitaxel.

1-8. (canceled)

9. A method for preparing polymeric nanoparticles, wherein said method comprises a double emulsion process comprising:
(a) dissolving a pharmaceutically acceptable PLGA polymer in a first solvent to form a polymer solution and dissolving or dispersing an active pharmaceutical ingredient (API) in a second solvent comprising water;
(b) adding a small amount of the second solvent to the polymer solution to form a mixture, wherein the first solvent is not miscible or only partially miscible with the second solvent;
(c) emulsifying the mixture by microfluidization to form a first emulsion;
(d) emulsifying the first emulsion in a third solvent comprising water and a surfactant to form a second emulsion; and,
(e) removing the first solvent to form said nanoparticles, wherein said nanoparticles have an Z average particle size of about 100 nm or less.

10. (canceled)

11. The method of claim 9, wherein the API is soluble in the first solvent.

12. (canceled)

13. (canceled)

14. The method of claim 9, wherein the API is a biologic entity.

15. The method of claim 14, wherein the biologic entity is a protein.

16-23. (canceled)

24. The method of claim 9, wherein the surfactant is polyvinyl alcohol.

25. (canceled)

26. (canceled)

27. The method of claim 9, wherein the microfluidization is performed at an applied pressure between 1,000-70,000 psi.

28. The method of claim 27, wherein the microfluidization is performed at a flow rate between 1 mL/min-1 L/min.

29. The method of claim 27, wherein the emulsion is cycled through the microfluidizer 2-10 times.

30-35. (canceled)

36. The method of claim 9, wherein the first solvent is ethyl acetate or methylene chloride or chloroform, or combination thereof.

37. The method of claim 9, wherein the first solvent is mixed with a co-solvent miscible with water.

38-42. (canceled)

43. The method of claim 9, wherein the polymer solution has a concentration between 10 mg/mL-100 mg/mL percent by weight.

44-47. (canceled)

48. The method of claim 9, wherein the first aqueous solution is added to the polymer solution in an amount of about 0.05 to about 25% (v/v).

49. The method of claim 9, wherein the first aqueous solution is added to the polymer solution in an amount of about 0.1 to about 10% (v/v).

51. The method of claim 9, wherein the nanoparticles have a Z average particle size from about 20 nm to about 90 nm.

52. The method of claim 29, wherein the first and second emulsions are cycled through the microfluidizer 2 to 10 times.
